(19) United States
(12) Patent Application Publication
(10) Pub. No.: US 2020/0330380 A1
(43) Pub. Date: Oct. 22, 2020

(54) BIOCOMPATIBLE ORGANOGEL MATRICES FOR INTRAOPERATIVE PREPARATION OF A DRUG DELIVERY DEPOT

(71) Applicant: DePuy Synthes Products, Inc., Raynham, MA (US)

(72) Inventors: Charles Florek, Downingtown, PA (US); David A. Armbruster, West Chester, PA (US); Sean Hamilton Kerr, Oreland, PA (US); Sanjay Jain, Chester Springs, PA (US); Junior Julien, West Chester, PA (US); Malavosklish Bikram-Liles, Paoli, PA (US)

(21) Appl. No.: 16/851,177

(22) Filed: Apr. 17, 2020

Related U.S. Application Data
(60) Provisional application No. 62/835,556, filed on Apr. 18, 2019.

(51) Int. Cl. A61K 9/06; A61K 47/14; A61K 47/12; A61K 47/10; A61K 47/26; A61K 9/14; A61K 31/7036; A61K 38/14; A61K 31/407; A61K 31/7032; A61K 9/00

(52) U.S. Cl. CPC A61K 9/06; A61K 47/14; A61K 47/12; A61K 47/10; A61K 47/26; A61K 9/0019; A61K 31/7036; A61K 38/14; A61K 31/407; A61K 31/7032; A61K 9/145

(57) ABSTRACT

The present disclosure is directed to an organogel drug depot for use in delivering an active agent to a surgical site, such as an implant site, for instance an orthopedic implant site. The present disclosure is also directed to an organogel drug depot for use in a non-sterile environment and application to a non-sterile open wound site. In a further embodiment, there is disclosed a system for preparing an organogel drug depot including an organogel matrix comprising an organogelator and a biocompatible organic solvent, an active agent comprising solid particles, a container including at least one wall having an outer surface and defining a volume capable of containing the organogel matrix and active agent solid particles, and a heating component configured to contact the outer surface and supply an amount of heat to the container.

### CROSS REFERENCE TO RELATED APPLICATIONS

This application claims the benefit of U.S. Provisional Application No. 62/835,556, filed on Apr. 18, 2019, the contents of which are hereby incorporated by reference in their entirety.

### FIELD OF DISCLOSURE

The present disclosure is directed to the perioperative and intraoperative preparation and delivery of organogel matrix drug delivery depots for local delivery of active agents to a surgical site or traumatic wound. More particularly, embodiments of the present disclosure are directed to preparation and local delivery of antimicrobial or anesthetic drug depots to a surgical site including one or more implantable medical devices, such as implantable orthopedic medical devices. The present disclosure is further directed to the preparation of a local drug depot formed from an organogel matrix in a non-sterile environment, and the application thereof to a non-sterile open wound.

### BACKGROUND

Foreign bodies, such as orthopedic implants, are a risk factor for postsurgical infection. References to antibiotic and antimicrobial eluting devices are plentiful in the literature, but commercially-available devices are rare. Bone cements, such as poly(methyl methacrylate) (PMMA) and calcium sulfate cements are used on and off label to deliver antibiotics to orthopedic surgical sites.

PMMA cement is non-resorbable and its use necessitates a removal operation. Additionally, the amount of PMMA needed for anti-infective therapy is especially disadvantageous in orthopedic applications due to limited soft tissue availability (i.e., limited volume for placement). Incomplete elution of antibiotics from PMMA cements results in uncertainty of dose. Furthermore, long-term low-dose delivery can lead to antibiotic resistance development. Additionally, the implanted PMMA material (e.g., beads) presents another foreign body for bacterial colonization and growth.

Calcium sulfate cement can be used as an antibiotic delivery reservoir in bone defects or in soft-tissue surrounding an orthopedic surgical site. In the US, studies have shown that calcium sulfate-based antibiotic therapies fail to provide controlled release of antibiotics for more than 3 days.

Another existing infection treatment option used is a surgeon directly delivering powdered antibiotic into the surgical site. Direct application of vancomycin powder in spine surgery was effective in case series, and a 1000 patient clinical trial has been conducted to measure the effect of local delivery of vancomycin on deep surgical site infections (SSIs) in high risk trauma surgery. Nevertheless, antibiotic powder application does not provide either sustained or controlled local tissue concentrations. Further, its use is limited to open surgical procedures, thus eliminating its treatment potential from percutaneous or minimally-invasive surgical procedures.

Hydrogels have also been considered as a delivery vehicle; however, their elution profiles are typically dominated by burst release with limited controlled, sustained release. Some examples include Novagenit's Defensive Antibacterial Coating (DAC) hydrogel, Dr. Reddy's laboratories' DFA-02, and Poloxamer 407 thermoreversible hydrogels. One study of Novagenit's DAC hyaluronan-poly-D,L-lactide hydrogel demonstrated that greater than 60% of vancomycin was released within the first 4 hours and that greater than 80% was released within 24 hours (Giavaresi G, Meani E, Sartori M, Ferrari, A, Bellini D, Sacchetta A C, Meraner J, Sambri A, Vocale C, Sambri V, Fini M, Romanó C L, International Orthopaedics (SCIOT) 2014; 38:1505-1512). A study of Dr. Reddy's DFA-02 gel reported results with a majority of antibiotic elution within 24 hours (Penn-Barwell J G, Murray K, and Wenke J C, J Orthop Trauma 2014; 28:370-375). A study of Poloxamer 407 thermoreversible hydrogel demonstrated extended vancomycin release in vitro; however, the local vancomycin concentration in a rat model at 24 and 48 hours was only 6% and 0.6% of the concentration at 4 hours demonstrating a significant decrease from initial release rates (Veyries M L, Couarraze G, Geiger S, Agnely F, Massias L, Kunzli B, Faurisson F, Rouveix B, International Journal of Pharmaceutics 1999; 192:183-193).

Sustained local release of antibiotics without removal of a device can be achieved with a bioresorbable antibacterial coating on a medical device; however, antibiotic coated devices in the orthopedic segment offer unique challenges. Many part numbers are required to fit patient anatomy, resulting in logistical challenges in coating, storing, and delivering sufficient stock of each size before expiration. Antibacterial implants would require a duplication of the inventory of the analogous non-antibacterial devices. Furthermore, the repeated sterilization of graphic cases is prohibitive to biodegradable antibacterial coatings, so alternate logistics are required.

Some difficulties associated with coated medical devices includes the limited market size per regulatory clearance, the necessity of duplicating inventory, and the technical challenge of coating the extensive varieties of anatomic implant shapes. Coated medical devices do not permit the surgeon to select desired antibiotics or combination of antibiotics. Evaluation of patient-specific risk factors or the species and sensitivities of bacteria recovered from patient tissues are important criteria in selecting the desired antimicrobial agents and dosage.

### SUMMARY

Accordingly, it would be beneficial to provide a drug depot that can be perioperatively or intraoperatively prepared and intraoperatively delivered to a surgical site, for instance a surgical site including one or more implantable medical devices, such as an implantable orthopedic medical device, where the drug depot is resistant to irrigation, resistant to migration from the surgical site and can provide controlled release of an active agent, such as an antimicrobial, antibiotic, or local anesthetic agent, or a combination thereof. In other words, the drug depot can remain at the surgical site for the duration of time necessary for the desired release of the active agent.

In additional embodiments, it would be beneficial to provide a drug depot that can be contemporaneously prepared and delivered to a non-sterile open wound site in a non-surgical setting; (i.e., a non-sterile environment), where the drug depot is migration resistant and can provide controlled release of an active agent, such as an antimicrobial agent or a local anesthetic. Such a drug depot that can be contemporaneously prepared and delivered can have particular advantage for use in acute emergency treatment settings with non-sterile open wounds involving significant soft and hard tissue damage, such as for use by emergency medical technicians or combat personnel, where the drug depot is contemporaneously prepared and delivered to the non-sterile open wound site. Such benefits include the ability to immediately deliver necessary anti-infective and pain relief treatment to a specific wound site of patient, where the drug depot is configured to remain at the site of delivery.

The present disclosure, therefore, in certain aspects, describes a method of delivering an active agent to a surgical site including the steps of:

perioperatively compounding solid particles of an active agent within a biocompatible organogel matrix so as to form an organogel drug depot configured for controlled release; and intraoperatively delivering the organogel drug depot to the surgical site; where the organogel matrix includes an organogelator and biocompatible organic solvent, and, where the organogel drug depot is in a solid or semisolid state during the step of intraoperative delivery.

According to certain embodiments, the surgical site can include one or more implantable medical devices, such as, for example, an implantable orthopedic device.

According to additional aspects of the present disclosure, a method of preparing a local drug depot having an active agent for delivery to a surgical site includes:

perioperatively compounding solid particles of an active agent within a biocompatible organogel matrix to form an organogel drug depot configured for controlled release;

where the organogel matrix comprises an organogelator and a biocompatible organic solvent, and where the organogel drug depot is in a solid or semisolid state prior to a delivery of the organogel drug depot.

According to certain embodiments, the surgical site can include one or more implantable medical devices, such as, for example, an implantable orthopedic device.

According to certain embodiments, compounding can include heating the organogel matrix to melt the matrix and incorporating the solid particles into the melted matrix. The method can further include, after incorporating the solid particles, cooling the melted matrix to form the organogel drug depot, where the drug depot is in a solid or semisolid state. In some embodiments, cooling the melted matrix occurs within about 10 minutes or less, for example, 5 minutes or less. In alternative embodiments, compounding can include a physical mixing (e.g., mechanical mixing) between the organogel matrix in the solid or semisolid state and the active agent solid particles to form the organogel drug depot, where the drug depot can be in a solid or semisolid state. In still further embodiments, compounding can include a combination of heating and physical or mechanical mixing.

According to certain embodiments, the organogel matrix has a solubility in water of less than 1 g/L.

According to certain embodiments, the organogel matrix has a melting point above 37° C. In certain embodiments, the organogelator includes one or more fatty acids or salts or esters of fatty acids, such as, for example, stearic acid, sodium stearate, or sorbitan monostearate, as well as mixtures thereof.

According to certain embodiments, the biocompatible organic solvent has a melting point below 20° C. According to further embodiments, the biocompatible organic solvent can include a biocompatible oil derived from a plant or animal, or synthetic derivatives thereof. In still further embodiments, the biocompatible oil includes one or more fatty acids. In still further embodiments, the one or more fatty acids can include unsaturated fatty acids, saturated fatty acids, or a combination or mixture thereof. In some embodiments, the one or more fatty acids can include free fatty acids, or can include fatty acids in the form of triglycerides, or a combination or mixture thereof. In one embodiment, the one or more fatty acids includes linoleic acid. Linoleic acid is a well-known component of a number of plant oils.

According to certain embodiments, the weight ratio of the organogelator and the biocompatible organic solvent of the organogel matrix is in the range of about 5:95 to about 60:40, such as, for example from about 25:75 to about 50:50.

According to certain embodiments, the active agent includes an antimicrobial agent, antibiotic agent, or a local anesthetic agent, or combination of the aforementioned active agents. According to certain embodiments, the active agent is soluble, freely soluble, or very soluble in water, as defined by the United States Pharmacopeia (USP) (i.e., a ratio of water to active agent of about 30:1 or less). In alternative embodiments, the active agent is sparingly soluble, slightly soluble, very slightly soluble, or insoluble in water, as defined by the USP (i.e., a ratio of water to active agent of about 30:1 or more).

According to certain embodiments, the solid particles of the active agent are disposed in within the organic solvent of the organogel matrix. In still further embodiments, the solid particles can have a median D(50) particle size (by volume distribution) in the range of about 1 μm to about 1 mm (1000 microns), such as, for example, in the range of about 1 μm to about 10 μm, or 10 μm to about 50 μm.

According to certain embodiments, the organogel matrix can further include one or more excipients. According to further embodiments, the one or more excipients includes biocompatible surfactants or biocompatible hydrophilic small molecules. In certain embodiments, the one or more excipients can include Poly(ethylene glycol) (PEG), Pluronic F127, Tween 80, or a mixture of any combination thereof.

According to certain embodiments, the organogel matrix is configured to adhere to a metal surface in an aqueous environment. This would include, for example, conditions simulating an in vivo aqueous environment.

According to certain embodiments, the surgical site is an implant site including one or more implantable medical devices, for instance, an implantable orthopedic device. In certain embodiments, an implantable medical device includes a metal surface, and the organogel matrix is configured to adhere to the metal surface in vivo. In certain embodiments, the organogel drug depot is intraoperatively delivered to the surgical site via percutaneous syringe injection, such as, for example, through incisions for screw placement in a percutaneous plating procedure. In additional embodiments, the surgical site (with or without an implantable medical device) is operatively opened and the drug depot is intraoperatively delivered to soft or hard tissue at the surgical site, and in procedures involving an implantable medical device at the surgical site, can be delivered adjacent to, or directly onto an outer surface of, an implantable medical device, such as, for example, a metal surface or an orthopedic implant. Typically, orthopedic implants include metal, polymer, or ceramic outer surfaces. In certain additional embodiments, the organogel drug depot is intraoperatively applied onto the implantable device outside the surgical site and then intraoperatively delivered to the surgical site with the implantable medical device.

According to the present disclosure, there is also described a system for preparing an organogel drug depot for local delivery to a surgical site. The system includes an organogel matrix including an organogelator and a biocompatible organic solvent, solid particles of an active agent, a container including at least one wall having an outer surface, where the container defines a volume capable of containing the organogel matrix and active agent solid particles, and a heating component configured to contact the outer surface and supply an amount of heat to the container.

According to certain embodiments, the surgical site is an implant site including one or more implantable medical devices, for instance, an implantable orthopedic device.

In certain embodiments of the system, the container is a syringe. In alternative embodiments, the container is a vial.

In still further embodiments, the system can include multiple containers, such that the container is a first container, and an additional container is a second container. In some embodiments, the first container has a first opening and the second container has a second opening, and the first opening is adapted to connect to the second opening.

In additional embodiments, the heating component defines an inner wall. Additionally, the inner wall can include, in some embodiments, at least one heating element, and further that the inner wall is configured to contact the outer surface of the container such that the at least one heating element supplies heat to the organogel matrix.

In certain embodiments, the inner wall defines a substantially cylindrical shape along its length. In still further embodiments, the inner wall defines a first cross-sectional diameter at a first region and a second cross-sectional diameter at a second region, and the first cross-sectional diameter can be greater than the second cross-sectional diameter.

In certain embodiments, the heating element is configured to provide one or more heating profiles along the inner wall, such that the heating component includes at least a first heating profile and a second heating profile.

According to still further embodiments of the present disclosure, methods of delivering an active agent to a non-sterile open wound site are described, including the steps of:

compounding solid particles of an active agent within a biocompatible organogel matrix to form an organogel drug depot; and,

delivering the organogel drug depot to a non-sterile open wound site, where at the time of delivery the open wound site includes soft tissue, hard tissue, or both, that are exposed to a non-sterile environment;

wherein the step of compounding and delivering are performed contemporaneously; and,

wherein the organogel is in a solid or semisolid state during the step of delivering.

According to additional aspects of the present disclosure, there is a method of preparing a local drug depot in a non-sterile environment for delivery of an active agent to a non-sterile open wound site including:

compounding solid particles of an active agent within a biocompatible organogel matrix to form an organogel drug depot;

wherein the step of compounding is performed contemporaneous to a delivery; and,

wherein the organogel is in a solid or semisolid state during compounding.

According to certain embodiments, contemporaneous compounding and delivery are within two hours or less of each other, for example within 1.5 hours, with 1.0 hours, or within 0.5 hours.

According to certain embodiments, the compounding comprises heating the organogel matrix to melt the matrix and incorporating the solid particles into the melted matrix. In further embodiments, the method further comprises, after incorporating the solid particles, cooling the melted matrix to form the organogel drug depot. In certain additional embodiments, cooling the melted matrix is about 10 minutes or less.

According to certain embodiments, compounding comprises a physical mixing between the organogel matrix in solid or semisolid state and the solid particles.

According to certain embodiments, the organogel matrix has a solubility in water of less than 1 g/L.

In certain embodiments, the organogel matrix is configured to adhere to the soft tissue, hard tissue, or both, in a substantially aqueous environment

According to certain embodiments, the active agent is an antimicrobial agent, antibiotic agent, or an anesthetic agent, or a combination thereof. In preferred embodiments, the active agent is selected from Cephalosporins, Aminoglycosides, Glycopeptides, Fluoroquinolones, Lipopeptides, Carbapenems, Rifamycins, as well as Antifungals, and combinations thereof. Suitable exemplary active agents can include cefazolin, cefuroxime, amikacin, gentamicin, tobramycin, vancomycin, ciprofloxacin, moxifloxacin, daptomycin, meropenem, ertapenem, rifampin, amphotericin-B, and fluconazole.

In additional embodiments, the active agent is soluble, freely soluble, or very soluble in water. According to alternative embodiments, the active agent is sparingly soluble, slightly soluble, very slightly soluble, or insoluble in water. In still further embodiments, the active agent solid particles have a D(50) median particle size distribution in the range of 1 μm to about 1 mm.

According to certain embodiments, the organogel matrix further comprises one or more excipients. In certain embodiments, the one or more excipients includes biocompatible surfactants or biocompatible hydrophilic small molecules, or a combination thereof. In still further embodiments, the one or more excipients includes Poly(ethylene glycol) (PEG), Pluronic F127, Tween 80, or a mixture of any combination thereof.

According to certain embodiments, the contemporaneous compounding and delivering are within 1.5 hours or less of each other. In still further embodiments, the contemporaneous compounding and delivering are within 1.0 hours or less, and can be within 0.5 hours or less.

### BRIEF DESCRIPTION OF THE DRAWINGS

The drawings illustrate generally, by way of example, but not by way of limitation, various embodiments discussed in the present disclosure. The foregoing summary, as well as the following detailed description of preferred embodiments of the application, will be better understood when read in conjunction with the appended drawings:

FIG. 1 is a front perspective view of a heating component according to one embodiment having a C-clip configuration;

FIG. 2A is a front perspective view of heating component according to another embodiment including an elastomeric step-tapered configuration;

FIG. 2B is a cross-section side view of the heating component of FIG. 2A;

FIG. 3 is a perspective view of another embodiment of a heating component having a hinge-shaped configuration;

FIG. 4A is a perspective view of heating device including a cradle shaped base unit with two connected syringes in an upright configuration and a drug-loading funnel;

FIG. 4B is a perspective view of the cradle-shaped heating device of FIG. 6A in a different configuration, including the heating component of FIG. 3 disposed in the base unit and retaining one of the syringes;

FIG. 4C is a cross-sectional view of the cradle-shaped base unit of FIG. 4A;

FIG. 5 is a front view of a heating device for use with a vial including a luer lock adapter cap;

FIG. 6A is a front perspective view of a heating device for use with a syringe and a stand including a heating component configured to attachably couple to a base unit with a drug-loading funnel;

FIG. 6B is a front perspective view of the heating device of 6A assembled for heating and melt-mixing;

FIG. 7 is a perspective view of a heating device including a heating component configured to attachably couple to a base unit;

FIG. 8A is a photograph of an organogel matrix that has been applied and adhered to the bottom of a metal weigh boat filled with phosphate buffered saline (PBS);

FIG. 8B is a photograph of three organogel matrix formulations that are adhered to the bottom of a metal weigh boat after exposure to a spray of deionized water;

FIG. 9A is a photograph showing the application of an organogel matrix including toluidine blue O dye applied onto a metal bone plate and surrounding tissue of a chicken thigh;

FIG. 9B is a photograph showing the applied organogel matrix of FIG. 9A after irrigation and manual rubbing of the bone plate with the skin closed over the plate;

FIG. 10A is a photograph showing the percutaneous injection of an organogel matrix including toluidine blue O dye applied through a skin incision of a chicken thigh;

FIG. 10B is a photograph showing distribution of the organogel matrix to the exposed muscle and fascia of the chicken thigh of FIG. 10A after percutaneous injection;

FIG. 10C is a cross section of muscle tissue recovered after a subcutaneous injection of organogel;

FIG. 10D is a photograph of organogel matrix containing toluidine blue O dye on chicken muscle and hypodermis tissue;

FIG. 11 is a photograph showing reconstitution of a semisolid organogel matrix from a melt state over the course of 5 minutes;

FIG. 12A is a differential scanning calorimeter graph showing temperature and heat values for an organogel matrix;

FIG. 12B is a differential scanning calorimeter graph showing temperature and heat values for the organogel matrix formulation of FIG. 12A including the addition of excipients;

FIG. 13 is a photograph of a battery-powered heating device melting 6 grams of organogel matrix in approximately 2 minutes;

FIG. 14A is a graph showing the 14 day cumulative release profiles of gentamicin sulfate from three organogel drug depot formulations mixed at room temperature;

FIG. 14B is a graph showing the 14 day cumulative release profiles of gentamicin sulfate from three melt-mixed organogel drug depot formulations;

FIG. 14C is a graph comparing the release profiles of the three organogel drug depot formulations of FIG. 14B against the release profiles from two published hydrogel systems;

FIG. 15 is a graph showing the 7 day cumulative release profiles of four melt-mixed organogel drug depots formulations; and,

FIG. 16 is a graph of the log reduction in colony forming units (CFU) of a 3-day staphylococcus aureus biofilm grown on an orthopedic implant from systemic levels of gentamicin versus gentamicin delivered from an organogel.

### DETAILED DESCRIPTION

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Further, reference to values stated in ranges includes each and every value within that range. It is also to be appreciated that certain features of the invention, which, for clarity, are described herein in the context of separate embodiments, may also be provided in combination in a single embodiment. Conversely, various features of the invention that are, for brevity, described in the context of a single embodiment, may also be provided separately or in any subcombination.

Descriptive terms related to the solubility of a given solute in a given solvent are made with reference to the use of those terms as understood and used by the United States Pharmacopeia (USP) as follows:

“Very Soluble” as used herein means less than one part of solvent is required for one part of solute. “Freely Soluble” as used herein means that from about 1 to about 10 parts of solvent is required for one part of solute. “Soluble” as used herein means that from about 10 to about 30 parts of solvent is required for one part of solute. “Sparingly Soluble” as used herein means that from about 30 to about 100 parts of solvent is required for one part of solute. “Slightly Soluble” as used herein means that from about 100 to about 1,000 parts of solvent is required for one part of solute. “Very Slightly Soluble” as used herein means that from about 1,000 to about 10,000 parts of solvent is required for one part of solute. “Practically Insoluble” or “Insoluble” as used herein means that greater than or equal to about 10,000 parts of solvent is required for one part of solute.

As used herein “semisolid” when used in describing properties of the organogel, means that the organogel matrix, or the organogel drug depot, does not flow without extrinsic application of force, yet the material will flow upon application of force, such as, for example, upon dispensing from a syringe or manual spreading across tissue within a surgical site. This definition includes, but is not limited to, Bingham plastics.

As used herein “melt” is the state change of the solid or semisolid organogel matrix or organogel drug depot to a liquid state.

As used herein, “organogelator” is a solid or semisolid organic compound defined by its monomeric subunit, which, when placed in contact with a biocompatible organic solvent, such as an oil, forms networks that act to stabilize the organic solvent, forming an organogel. In certain embodiments, the network is a three-dimensional fibrillar network.

As used herein, “organogel matrix” is a gel composed of at least an organogelator and a biocompatible organic solvent, such as an oil. The organogelator according to the present disclosure can further include one or more excipients. While it is commonly understood that an organogel matrix will typically constitute a majority percentage by weight of the biocompatible organic solvent, for the purpose of this disclosure, the organogel matrix described herein can, in some embodiments, include equal amounts of each component, and in further embodiments, the organogelator can be a majority constituent by weight.

As used herein, “intraoperative” means the time period during a surgical procedure.

As used herein, “perioperative” means the time frame during the course of a surgical procedure (i.e., intraoperative), as well as, a reasonable time period prior to the surgical procedure. For the purposes of this disclosure, a reasonable time period can be considered within six to eight hours of the surgical procedure.

As used herein “contemporaneous” means within 2 hours or less, such that the delivery of the organogel drug depot to the soft or hard tissue or both, will be within any time period within 2 hours or less from the start of the preparation of the organogel drug depot, for example, 1.5 hours, 1.0 hour, 45 minutes, 30 minutes, 20 minutes, or 10 minutes, or any range or combination of ranges within 2 hours or less.

As used herein “non-sterile” means an environment, location, or surface that is not free from viruses, bacteria, foreign bodies, or any other potentially infection causing components.

As used herein “open wound” means a traumatic injury where the skin is torn, cut, or punctured such that the dermis is damaged, and the underlying fascia, muscle, bone, or other internal organs are exposed to the external environment. Such open wounds can be the result of lacerations, abrasions, avulsions, punctures, or penetrations to the skin and can have a likelihood of contamination.

The present disclosure describes an organogel matrix containing solid particles of an active agent for use as a local drug depot at a surgical site. The disclosed organogel drug depot provides the advantage of a controlled release matrix that is biocompatible, hydrophobic, tissue-adherent, implant adherent, and migration resistant, can be injectable, or applied manually, and does not inhibit healing at the surgical site. The disclosed delivery process of the present disclosure has the further advantage of permitting the medical professional to select an active agent and release rate based upon an individual patient's specific needs and risk factors in contrast to pre-coated, or other types of pre-loaded, or fixed dose medical implants.

An additional advantage of the disclosed organogel drug depot and delivery process is that it permits the contemporaneous preparation and delivery to a non-sterile open wound site, such as an acute traumatic injury site (e.g., combat injury or machine accident) with desired adherence to the tissue at the wound site to achieve the necessary therapeutic effect, such as for example infection prevention or pain relief.

The organogel matrix has the advantages of low-temperature melting, tunable-release, and a variety of strategies for room temperature or melt reconstitution of active agent particles (e.g., Active Pharmaceutical Ingredient (API) powders) that enables the medical professionals to formulate an antibacterial, anesthetic, or other drug delivery depot perioperatively, and particularly intraoperatively. Moreover, the organogel matrices allow for application and retention to both hard and soft tissue surfaces, as well as metal surfaces in aqueous environments such as in vivo conditions. This permits implantable medical devices, such as implantable orthopedic devices to be coated with the organogel drug depot after completion of internal fixation and prior or subsequent to final irrigation before closure; or, alternatively to be coated with the organogel drug depot prior to implantation of the medical device, such that the delivery of the organogel drug depot and the implantable medical device to the surgical site occurs simultaneously.

For example, in certain embodiments, the organogel drug depot may be prepared within 15 minutes and is stable enough to allow for preparation up to at least 6-8 hours ahead of delivery to the surgical site. This allows for intraoperative or perioperative preparation of the organogel drug depot such that all available patient data can be included in the selection of the drug molecule and delivery duration at or near the time of delivery. It should be appreciated that, in certain other embodiments, the organogel matrix could be prepared in a time period prior to a perioperative time period, such as for example, a manufacturer of a organogel matrix could prepare the composition at an offsite location and ship the composition to the surgical location, which at that point the perioperative compounding of the organogel matrix with the solid particles of an active agent can then occur.

The organogel drug depot of the present disclosure can additionally provide sufficient duration of active agent delivery clinically-relevant to local prevention of bacterial colonization or pain relief; typically within the range of about 1-14 days, and have sufficient dose strength to protect both the tissue surrounding the surgical site, and where applicable any implantable medical devices at the surgical site, such as in the case where antimicrobials, antibiotics, or local anesthetics are the desired active agents of interest. For example, in certain embodiments, the organogel drug depot can be configured for acute dosing, such as for example, less than 6 hours, or less than 12 hours, or less than 1 day to about 1-3 days. In certain other embodiments, the organogel drug depot can be configured for an intermediate dosing period, such as for example, in the range of 4-7 days. In additional embodiments, the organogel drug depot can be configured for a longer-term dosing period, such as for example, 7-14 days. In still further additional embodiments, the organogel drug depot can be configured for an extended release dosing period of up to 3-4 weeks. It should be appreciated that in embodiments where multiple active agents are utilized in the organogel drug depot, the organogel drug depot can be configured to have multiple dosing profiles (e.g., acute and long term) based upon the release profile of the selected active agents compounded within the organogel drug depot. Additionally, the organogel drug depot of the present disclosure has a sufficiently low bulk mass to allow for standard surgical soft tissue closure techniques at the surgical site as compared to use of antibiotic loaded cements as previously described. Furthermore, the organogel matrix can permit controlled release of multiple active agents having different properties such as molecular weight, log P values, etc., that would typically result in different release profiles in vivo.

In yet further embodiments of the present disclosure, the organogel drug depot has a lower limit to its viscosity range that is sufficiently high such that without application of extrinsic force the organogel drug depot exhibits substantially no flow. Furthermore, the organogel drug depot has an upper limit to its viscosity range that is sufficiently low such that application of mechanical force (e.g., a hand or surgical tool or device) to the organogel drug depot permits the even spreading or distribution (i.e., shearing) of the organogel drug depot to the necessary locations in and around the surgical site, such as the soft or hard tissues, or any implantable medical devices at the surgical site.

According to the present disclosure, a method of delivering an active agent to a surgical site is described including the steps of:

perioperatively compounding solid particles of an active agent within a biocompatible organogel matrix so as to form an organogel drug depot configured for controlled release; and intraoperatively delivering the organogel drug depot to the surgical site; where the organogel matrix includes an organogelator and a biocompatible organic solvent; and, where the organogel drug depot is in a solid or semisolid state during the step of intraoperative delivery.

According to embodiments of the present disclosure, the organogel matrix includes an organogelator and a biocompatible organic solvent. In certain embodiments, the organogelator is from a category of organogelator known as low molecular-mass organic gelators (LMOGs). LMOGs are characterized by their ability to form self-assembled gel networks, such as for example, fibrillar networks. The ability to self-assemble can occur from the formation of non-covalent interactions between the individual monomeric sub-units. According to certain embodiments, suitable organogelators can include fatty acids and derivatives thereof. For example, considering the fatty acid steric acid as an example, suitable embodiments would include stearic acid (fatty acid), sodium stearate (fatty acid salt), and sorbitan monostearate (fatty acid ester). Suitable organogelators can also include n-alkanes. In additional embodiments, suitable organogelators result in an organogel drug depot that has a melting point of at least about 37° C., and can, in certain embodiments, have a melting point as high as about 80° C.

According to certain embodiments, the biocompatible organic solvent is an organic solvent approved for use in humans by the U.S. Food and Drug Administration. In certain embodiments, the biocompatible organic solvent is a plant or animal based oil or a synthetic derivative thereof. In certain embodiments, the oil includes one or more fatty acids. In still further embodiments, the one or more fatty acids can include unsaturated fatty acids, saturated fatty acids, or a combination or mixture thereof. In some embodiments, the one or more fatty acids can include free fatty acids, or can include fatty acids in the form of triglycerides, or a combination or mixture thereof. In one embodiment, the one or more fatty acids includes linoleic acid, which, for example, is a main component of cotton seed oil. In still further embodiments, the oil has a melting point below 20° C.

According to certain embodiments, the active agent is an antimicrobial agent, antibiotic agent, or an anesthetic agent, or a combination thereof. In preferred embodiments, the active agent is selected from Cephalosporins, Aminoglycosides, Glycopeptides, Fluoroquinolones, Lipopeptides, Carbapenems, Rifamycins, as well as Antifungals, and combinations thereof. Suitable exemplary active agents can include cefazolin, cefuroxime, amikacin, gentamicin, tobramycin, vancomycin, ciprofloxacin, moxifloxacin, daptomycin, meropenem, ertapenem, rifampin, amphotericin-B, and fluconazole. Suitable anesthetic agents can include, for example, benzocaine, proparacaine, tetracaine, articaine, dibucaine, lidocaine, prilocaine, pramoxine, dyclonine, and bupivacaine.

According to certain embodiments, the active agent is soluble, freely soluble, or very soluble in water, as defined by the United States Pharmacopeia (USP). In alternative embodiments, the active agent is sparingly soluble, slightly soluble, very slightly soluble, or insoluble in water, as defined by the USP.

According to certain embodiments, the solid particles of the active agent are disposed within the organic solvent component of the organogel matrix. In still further embodiments, the solid particles can have a D(50) median particle size (by volume distribution) in the range of about 1-1000 μm, such as, for example, in the range of about 1 μm to about 10 μm, about 1 μm to about 5 μm, about 5 μm to about 10 μm, about 10 μm to about 20 μm, about 10 μm to about 50 μm, about 1 μm to about 50 μm, about 50 μm to about 100, about 1 μm to about 100 μm, about 100 μm to about 500 μm, or about 100 μm to about 1000 μm.

In certain embodiments, the organogel drug depot has an active agent content in the range of about 1% to 30% by weight. In certain embodiments, the active agent content can be in the range of 1% to 5%, 1% to 10%, 5% to 10%, 10% to 20%, 5% to 20%, 10% to 30%, 20% to 30%, about 10%, about 20%, or about 25%, for example, or any combination of ranges listed above.

According to certain embodiments, the organogel matrix is very slightly soluble or insoluble in water, such that, for example, the organogel matrix has a solubility in water of less than lg/L. According to further embodiments, the organogel matrix can have a weight ratio of organogelator to biocompatible organic solvent in the range of about 5:95 to about 70:30. In still further embodiments, the weight ratio can be in the range of about 30:70 to about 50:50. For example the weight ratio can be 10:90, 25:75, 30:70, 40:60, 45:55, 50:50, 55:45, 60:40, or 70:30.

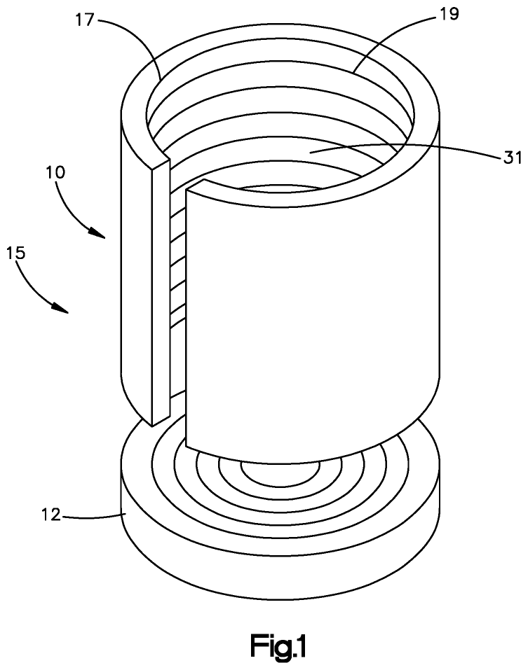

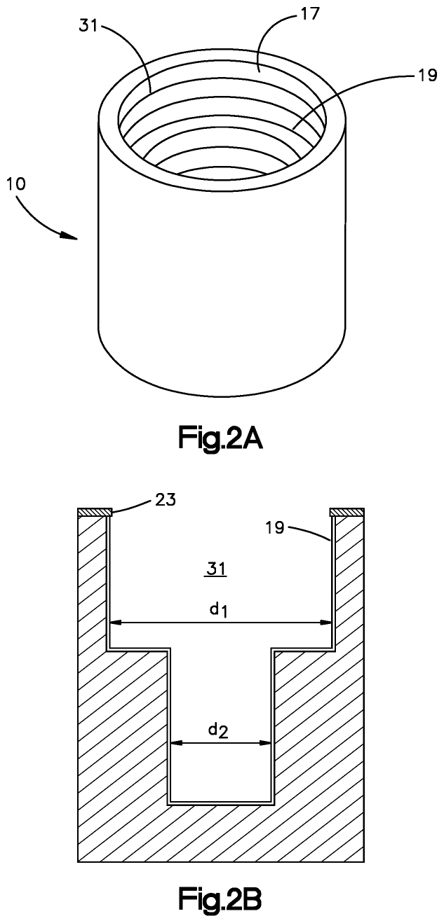

According to the present disclosure, and with reference to FIGS. 1-2, in certain embodiments, compounding can include heating the organogel matrix to melt the matrix and incorporating (e.g., suspending) the solid particles into the melted matrix. The method can further include, after incorporating the solid particles, cooling the melted matrix to form the organogel drug depot, where the drug depot is in a solid or semisolid state. In further embodiments, perioperative compounding is intraoperative compounding. In some embodiments, cooling the melted matrix occurs within about 10 minutes or less, for example, 5 minutes or less. In alternative embodiments, compounding can include a physical mixing between the organogel matrix in solid or the semisolid state and the solid particles to form the organogel drug depot, where the drug depot is in a solid or semisolid state. In still further embodiments, compounding can include a combination of heating and physical mixing.

According to certain embodiments, the organogel matrix can further include one or more excipients. In certain embodiments, the one or more excipients includes biocompatible surfactants or biocompatible hydrophilic small molecules, or a combination thereof. In still further embodiments, the biocompatible hydrophilic small molecules can increase the water-solubility of the matrix. In further embodiments, the small molecule has a weight average molecular weight of about 20,000 Daltons (20 kD) or less. In certain embodiments, the one or more excipients can include PEG_{10 k}, Pluronic F127, Tween 80, or a mixture of any combination thereof.

According to certain embodiments, the organogel drug depot is intraoperatively delivered to the surgical site via percutaneous syringe injection through a cannula. In additional embodiments, the surgical site (with or without an implantable medical device) is operatively open and the drug depot is intraoperatively delivered onto soft or hard tissue at the surgical site. In procedures including one or more implantable medical devices, the intraoperative delivery of the organogel drug depot can additionally include delivery adjacent to, or directly onto, an outer surface of an implantable medical device, such as, for example, a metal surface or an orthopedic implant. In certain additional embodiments, the organogel drug depot is first intraoperatively applied onto the implantable device outside the surgical site and then intraoperatively delivered to the surgical site with the implantable medical device.

According to the present disclosure, there is also described a system for preparing an organogel drug depot for local delivery to a surgical site or as has also been described, for local delivery to a non-sterile open wound site. The system includes an organogel matrix including an organogelator and an oil, solid particles of an active agent, a container including at least one wall having an outer surface, where the container defines a volume capable of containing the organogel and active agent solid particles, and a heating element configured to contact the outer surface and supply an amount of heat to the container.

In certain embodiments of the system, the container is a syringe. In alternative embodiments, the container is a vial. In certain instances, the container can be formed specifically to compliment the shape of a heating component. In certain other instances, the vial can be the original drug manufacture vial.

In still further embodiments, the system can include multiple containers, such that the container is a first container, and an additional container is a second container. In some embodiments, the first container has a first opening and the second container has a second opening, and the first opening is adapted to connect to the second opening.

With reference to FIGS. 1-3, a heating component 10 is disclosed, the heating component 10 defining an inner wall 17. The inner wall 17 can include, in some embodiments, at least one heating element 19, and further that the inner wall 17 is configured to contact the outer surface of the container (not shown) such that the at least one heating element 19 supplies heat to the organogel matrix.

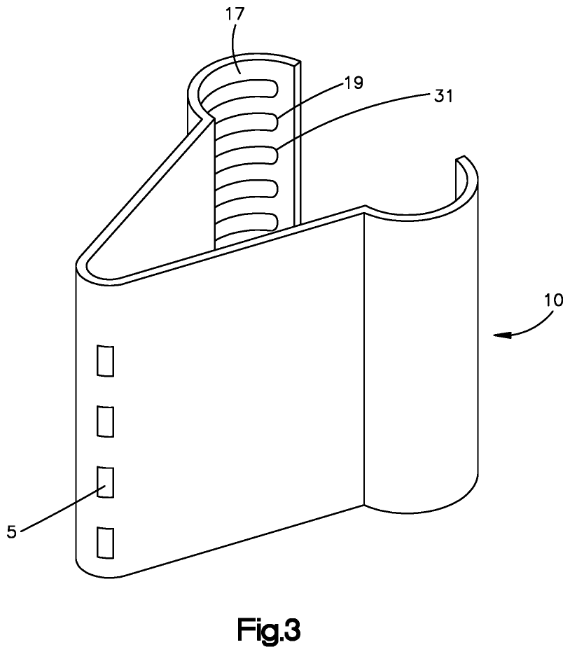

In certain embodiments, such as is shown in FIGS. 1 and 3, the inner wall 17 defines a substantially uniform cylindrical shape along the length of the heating component 10. In still further embodiments, such as is shown in FIGS. 2A-B, the inner wall 17 can define a non-uniform cross section, such that for example, the inner wall 17 defines a first cross-sectional diameter d₁ at a first region and a second cross-sectional diameter d₂ at a second region, and the first cross-sectional diameter can be greater than the second cross-sectional diameter.

In certain embodiments, the heating element 19 is configured to provide a uniform heating profile substantially along the length of the heating component 10. In other embodiments, the heating element 19 is configured such that it can provide one or more heating profiles along the inner wall 17, such that the heating device 10 includes at least a first heating profile and a second heating profile.

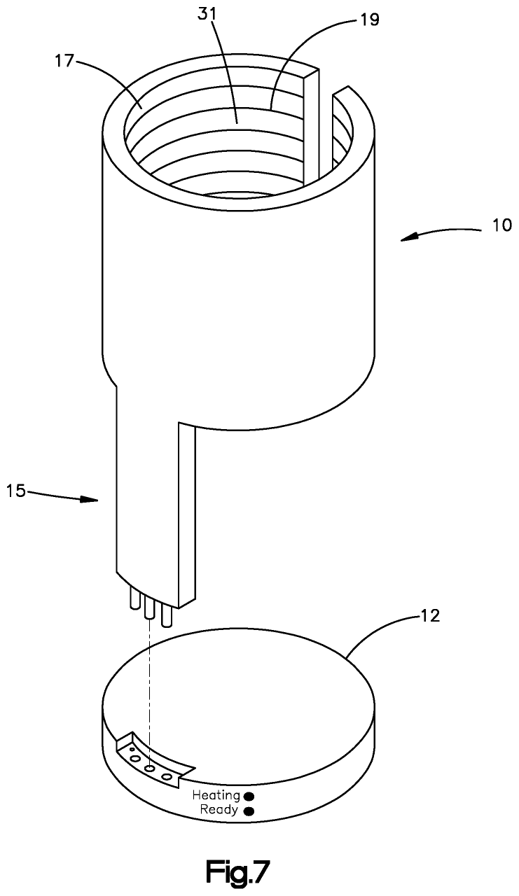

Referring to FIG. 1, a heating device 15 is shown including a heating component 10 configured in the shape of a C-clip, and a base unit 12. According to certain embodiments, and as shown in FIG. 1, heating component 10 and base unit 12 are integrally formed into a monolithic heating device 15. In alternative embodiments, such as shown in FIG. 7, heating component 10 and base unit 12 are configured such that heating component 10 can attachably couple to base unit 12. Base unit 12 can, in certain embodiments, house a power supply and electronics necessary to supply energy to the heating component and to configure one or more heating profiles for the heating component 10. In certain other embodiments, the base unit 12 is optional, such that the heating device 15 consists only of the heating component 10. In these embodiments, the heating component 10 can provide its own power to generate heat. The heating component 10, according to one embodiment defines a substantially cylindrical shaped inner wall 17 along its length that includes one or more heating elements 19 disposed along the length of its surface. The inner wall 17 defines a cavity 31 shaped to accept a container (not shown), such as for example, a syringe or a vial. Because the heating component 10 has a C-clip configuration, which can rely on a snap-fit or friction-fit engagement with the container, it can accommodate containers having a range of cross-sectional diameters.

Referring to FIGS. 2A-B, a heating component 10 is shown configured in the shape of a tiered chamber. The heating component 10 further defines an inner wall 17 including one or more heating elements 19 along its length. The inner wall 17 defines a cavity 31 having one or more cross-sectional diameters along its length such that the heating component 10 can include a first cross-sectional diameter d₁ at a first region and a second cross-sectional diameter d₂ at a second region, and wherein the first cross-sectional diameter is greater than the second cross-sectional diameter. The heating component 10 is therefore configured, according to certain embodiments, to accept containers (not shown) having a smaller cross-section diameter in the second region, and accept containers having a greater cross-sectional diameter in the first region. The heating component 10 can further include, in certain embodiments, one or more lips 23 that extend into the cavity region 31 such that the lips are adapted to secure the container, for example, by a friction fit or other mechanical restraint.

Referring to FIG. 3, a heating component 10 is shown configured in the shape of a living hinge (or clamshell hinge). The heating component 10 further defines an inner wall 17 including one or more heating elements 19. The inner wall 17 defines a cavity 31 shaped to accept and secure a container (not shown) through a mechanical friction fit. Because the heating component 10 is configured in the shape of a hinge, it can accommodate containers having a range of cross-sectional diameters.

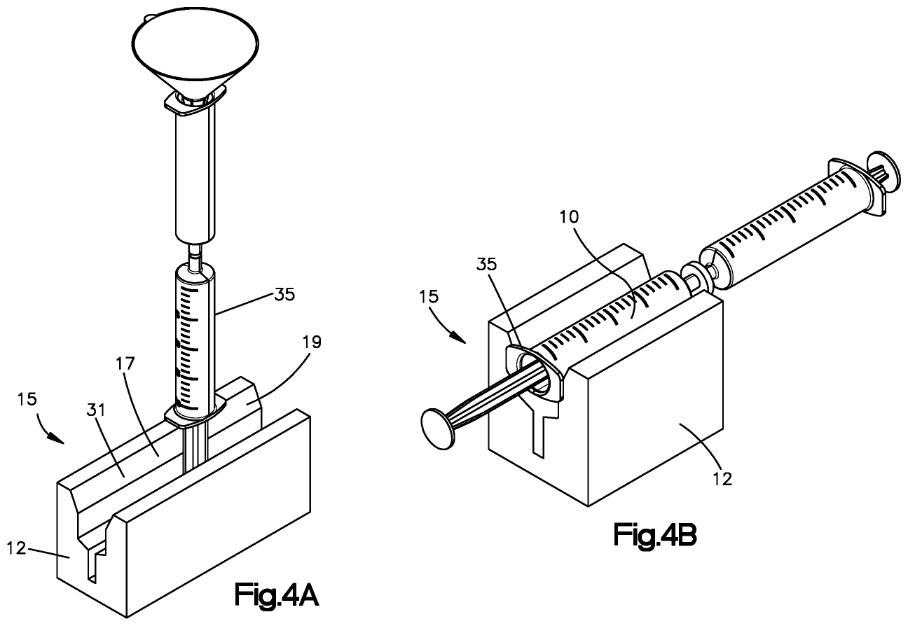

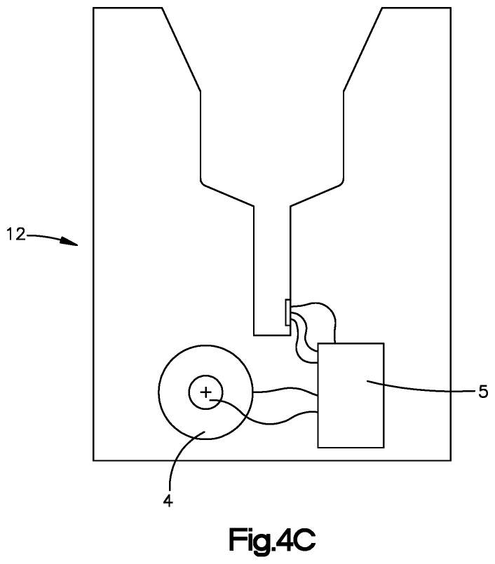

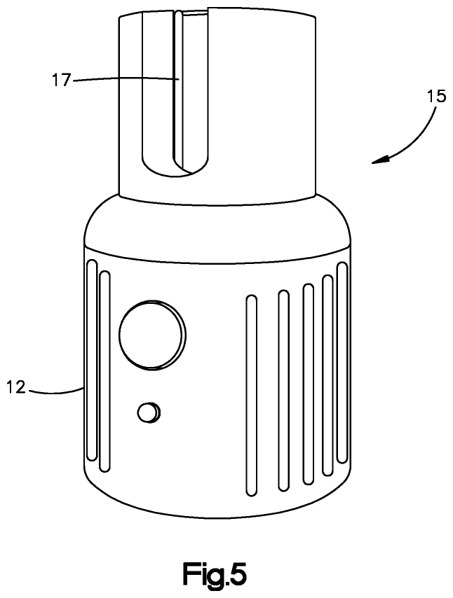

Referring to FIGS. 4A-C, a heating device 15 is shown having a base unit 12 configured in the shape of an elongated cradle. According to certain embodiments, as shown in FIG. 4A, the heating device 15 further includes a heating component 10 integrally formed with base unit 12 such that the heating component 10 and base unit 12 form a single integral body. Heating device 15 further defines an inner wall 17 including one or more heating elements 19. The inner wall 17 defines a cavity 31 shaped to accept a container 35. Further, as shown in FIG. 4A, the inner wall 17 of the device body 15 is dimensioned to allow a container 35 (shown here as a syringe) to be secured in an upright position to allow for the container 35 to be filled with either the organogel matrix, the active agent, or both.

According to certain embodiments, as shown in FIG. 4B, the heating device 15 can include a base unit 12 configured in the shape of a cradle, where the base unit 12 is dimensioned to allow heating component 10 (as shown here, the hinged heating component of FIG. 3) to attachably couple to base unit 12. Additionally, as shown, the inner wall 17 of heating component 10 is dimensioned to allow the container 35 to be positioned within the cavity 31 such that the container 35 is in contact with the heating elements 19 of the inner wall 17 of the heating component 10. FIG. 4C shows one embodiment of the base unit 12 housing a battery 4 and the corresponding electronics 5 utilized to provide energy to the heating component 10 when base 12 and heating component 10 are operatively coupled together.

Referring to FIG. 5, a heating device 15 is shown including a heating component (not shown) integrally formed with base 12. Inner wall 17 defines a cavity (not shown) to receive a container (not shown). Additionally, the heating device 15 can include a luer lock adapter cap system to facilitate the connection of a first container, for example, a vial, to a second container, for example, a syringe. It should be appreciated that heating component 10 could be detachably coupled to base 12, such as for example, the heating components shown in FIGS. 1-2, being slidably inserted into base 12, in order to accommodate a container having a corresponding shape as desired.

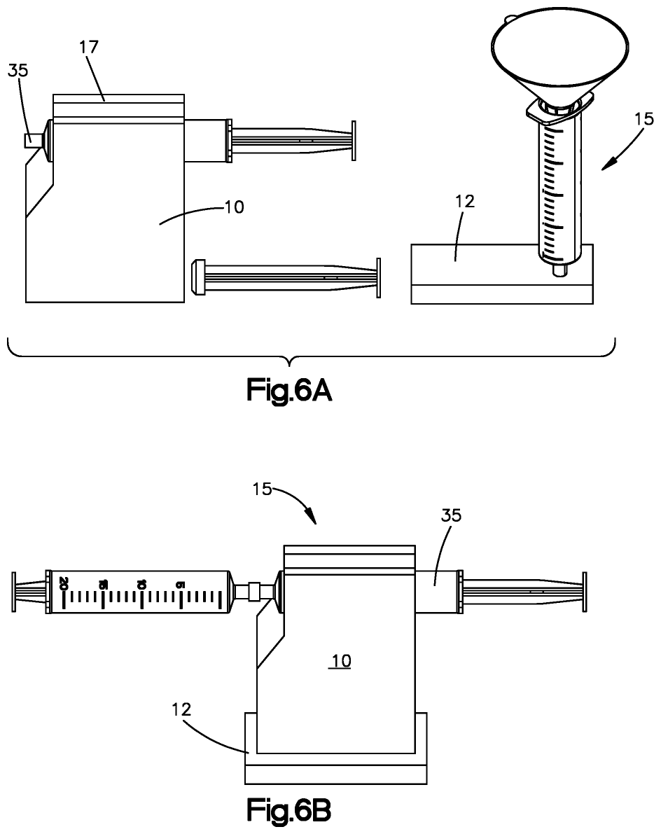

Referring to FIGS. 6A-B, a heating device 15 is shown including a heating component 10 and base unit 12. As shown in FIG. 6A, heating component 10 is detached from base 12. Heating component 10 includes an inner wall 17 defining a cavity (which as shown here, is occupied with container 35, shown as a syringe). The container 35 is in contact with heating elements 19 (not shown) disposed along the inner wall. Heating component 10, according to certain embodiments, and as shown here, can be shaped and dimensioned to include batteries 4 (not shown but contained within) to supply power. Base 12, can include in certain embodiments, a stand or mounting aid, for container 35 to assist a user in preparing the organogel compositions. Base 12 can further include the necessary electronics 5 for providing one or more heating profiles to the heating elements 19. As shown in FIG. 6B, base 12 and heating component 10 are connected such that a heating profile can be delivered to container 35 disposed within cavity 31.

Referring to FIG. 7, a heating device 15 is shown having a heating component 10 and base unit 12 that can be attachably coupled. Base unit 12 can include a power supply and the necessary electronics to provide one or more heating profiles to heating component 10. The heating component further defines an inner wall 17 including one or more heating elements 19. The inner wall 17 defines a cavity 31 shaped to accept and secure a container (not shown). The heating device 15 can be configured such that the base unit 12 provide a heating profile to the heating component 10 when they are operatively coupled. Alternatively, the base unit 12 can charge the heating component 10 with sufficient power such that heating component 10 can heat the container if it is detached from base unit 12. In other words, the heating component 10 can be portable and separable from the base unit 12 and still provide heat to the container.

According to the present disclosure, methods of delivering an active agent to a non-sterile open wound site are described including

compounding solid particles of an active agent within a biocompatible organogel matrix to form an organogel drug depot; and,

delivering the organogel drug depot to an open wound site, wherein at the time of delivery the open wound site includes soft tissue, hard tissue, or both, that are exposed to a non-sterile environment;

wherein the step of compounding and the step of delivering are performed contemporaneously; and,

wherein the organogel is in a solid or semisolid state during the step of delivering.

According to other embodiments of the present disclosure, methods of preparing a local drug depot in a non-sterile environment for delivery of an active agent to a non-sterile open wound site comprising:

compounding solid particles of an active agent within a biocompatible organogel matrix to form an organogel drug depot;

wherein the step of compounding is performed contemporaneous to a delivery; and, wherein the organogel is in a solid or semisolid state during compounding.

According to certain embodiment the contemporaneous compounding and delivering are performed within any time period within 2 hours or less from the start of the preparation of the organogel drug depot, for example, 1.5 hours, 1.0 hour, 45 minutes, 30 minutes, 20 minutes, or 10 minutes, or any range or combination of ranges within 2 hours or less.

According to certain embodiments, the open wound site can include exposed soft tissue, hard tissue, and fascia, as well as other underlying internal organs, the surfaces of which each are suitable for delivery of the organogel drug depot.

It should be appreciated that the previously disclosed components of the organogel drug depot, its properties, apply equally to this method of treatment of preparing and delivering an active agent to a non-sterile open wound site.

As such, according to certain embodiments, contemporaneous compounding and delivery are within two hours or less of each other, for example within 1.5 hours, with 1.0 hours, or within 0.5 hours.

According to certain embodiments, the compounding comprises heating the organogel matrix to melt the matrix and incorporating the solid particles into the melted matrix. In further embodiments, the method further comprises, after incorporating the solid particles, cooling the melted matrix to form the organogel drug depot. In certain additional embodiments, cooling the melted matrix is in about 10 minutes or less.

According to certain embodiments, compounding comprises a physical mixing between the organogel matrix in solid or semisolid state and the solid particles.

According to certain embodiments, the organogel matrix has a solubility in water of less than 1 g/L.

In certain embodiments, the organogel matrix is configured to adhere to the soft tissue, hard tissue, or both, in a substantially aqueous environment

According to certain embodiments, the active agent is an antimicrobial agent, antibiotic agent, or an anesthetic agent, or a combination thereof. In certain embodiments, the antibiotic agent is gentamicin or vancomycin. In additional embodiments, the active agent is soluble, freely soluble, or very soluble in water. According to alternative embodiments, the active agent is sparingly soluble, slightly soluble, very slightly soluble, or insoluble in water. In still further embodiments, the active agent solid particles have a D(50) median particle size distribution in the range of 1 μm to about 1 mm.

According to certain embodiments, the organogel matrix further comprises one or more excipients. In certain embodiments, the one or more excipients includes biocompatible surfactants or biocompatible hydrophilic small molecules, or a combination thereof. In still further embodiments, the one or more excipients includes Poly(ethylene glycol) (PEG), Pluronic F127, Tween 80, or a mixture of any combination thereof.

According to certain embodiments, the contemporaneous compounding and delivering are within 1.5 hours or less of each other. In still further embodiments, the contemporaneous compounding and delivering are within 1.0 hours or less, and can be within 0.5 hours or less.

### EXAMPLES

Metal Adherence

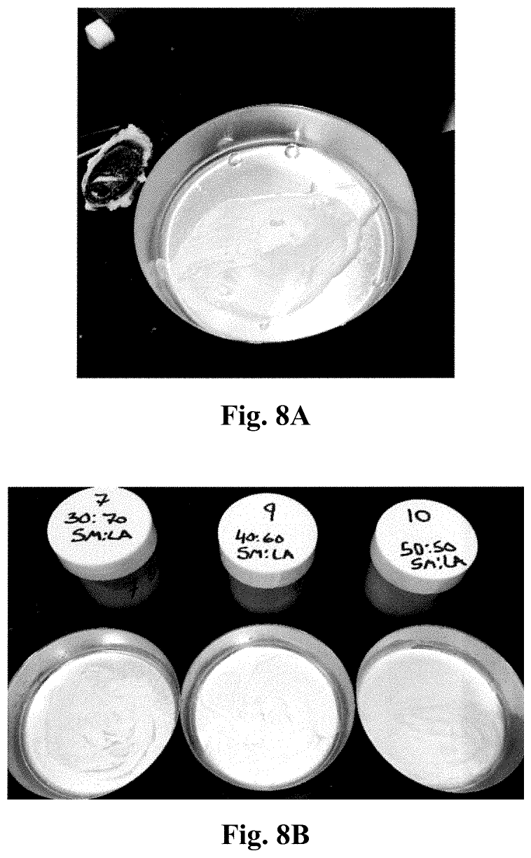

An application of 50:50 sorbitan monostearate:linoleic acid organogel matrix was applied onto the bottom surface of a metal weigh boat through an aqueous medium of phosphate buffered saline (PBS), as shown in FIG. 8A. The organogel matrix exhibited good adherence to the metal surface of the weigh boat through the aqueous medium of the PBS.

In a separate experiment, three different organogel matrix formulations were applied to the bottom surface of a metal weigh boat. The organogel formulations were composed of 30:70, 40:60, and 50:50 sorbitan monostearate:linoleic acid respectively. Each formulation was forcefully rinsed with deionized water from a squirt bottle to simulate aqueous conditions and fluid flow that can occur in vivo. The water stream did not dislodge the 40:60 and 50:50 organogel matrix formulations, while some of the 30:70 sorbitan monostearate:linoleic acid organogel matrix was dislodged but a visually-apparent quantity remained, which can be seen in FIG. 8B.

These results indicate the organogel matrix formulations of the present disclosure can be applied to metal surfaces, such as implantable medical devices like orthopedic implants in wet environments. Thus, the methods described herein can permit the organogel drug depots to be applied to the implantable medical device in vivo after completion of internal fixation, as well as prior to or subsequent to final irrigation before closure of the orthopedic implant site. It is further noted, that the solid/semisolid state of the organogel matrix at the time of delivery is sufficiently important to prevent the migration of the matrix away from the intended site and achieve good adherence to the desired surface.

Ex vivo application to simulated orthopedic implant site

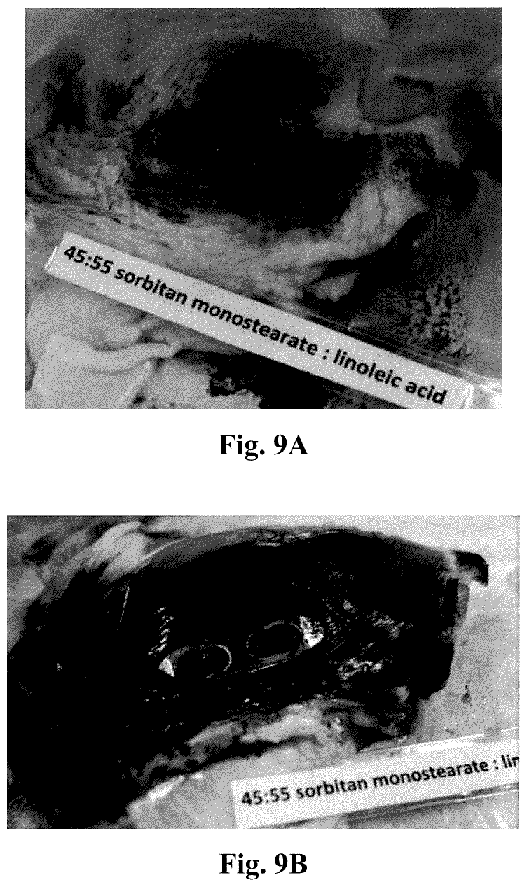

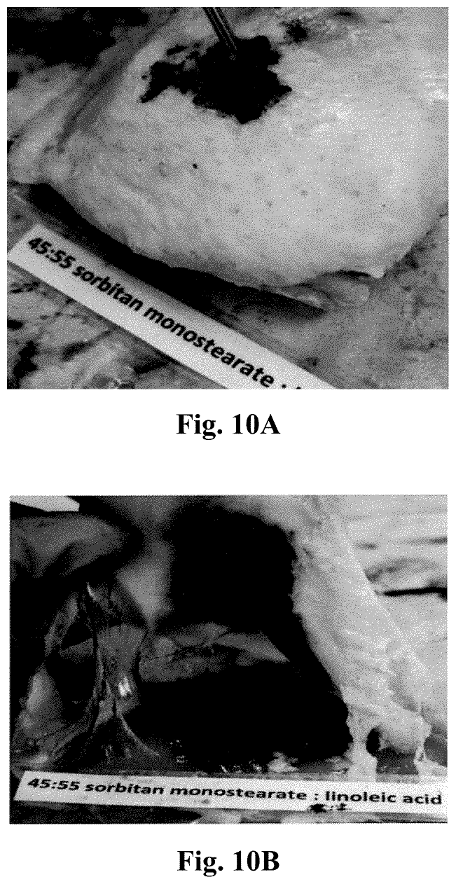

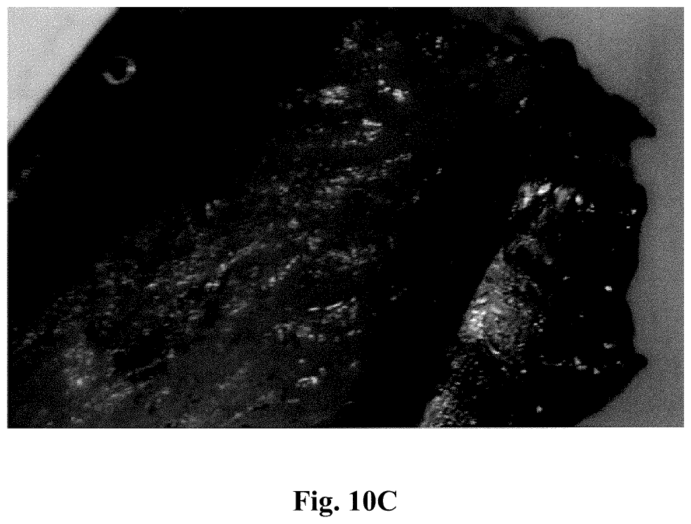

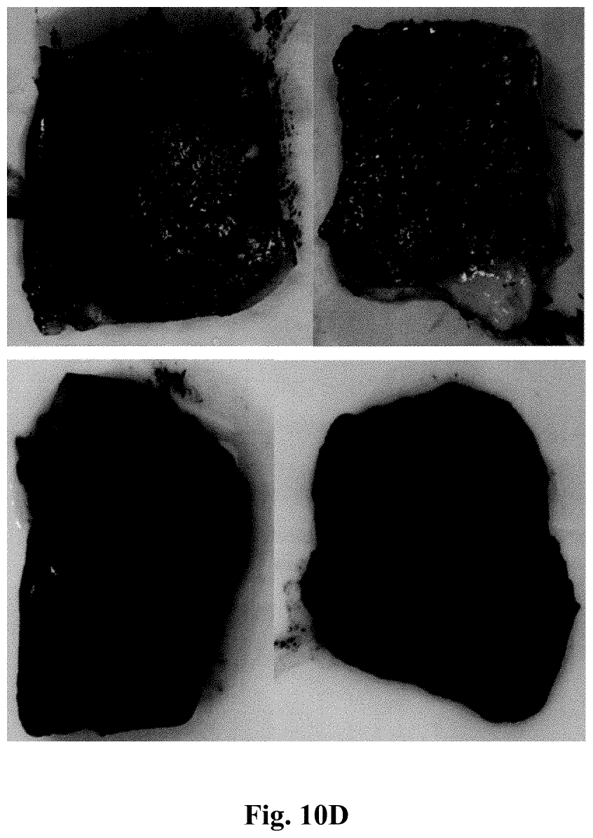

A 45:55 sorbitan monostearate:linoleic acid organogel matrix was loaded with toluidine blue O dye (to simulate a hydrophilic active agent) and was applied as a simulated organogel drug depot to orthopedic implant sites on chicken thighs. One site was used for open application along with a stainless steel plate, as shown in FIGS. 9A-B. A second site was used for percutaneous injection of the organogel drug depot at the simulated orthopedic implant site, as shown in FIGS. 10A-B. In open application of the organogel (FIGS. 9A-B), it was noted that the organogel matrix was adherent to the hypodermis-contaminated stainless steel plate, the muscle fascia, and the hypodermis even after irrigation with saline and manual rubbing of the site. The percutaneous simulated surgical site (FIGS. 10A-B) demonstrated the ability to cover a 40 cm² area through a single incision with adhesion to both hypodermis and muscle fascia.

It is believed that the semisolid nature of the organogel matrix permits it to be sheared over a large area without compromising the overall matrix; without being bound to any particular theory, this can be facilitated by weak associations between particles or self-assembled structures that stabilize the semisolid. The semisolid nature of the organogel matrix appears to prevent penetration of the matrix into adjacent tissue structures, as shown in FIG. 10C (noting that the organogel adheres to the fascia of the muscle but does not penetrate the muscle), while permitting the eluted drug to effectively release from the matrix and penetrate the adjacent tissue. Such results demonstrate the ability of the organogel matrix—and by extension, the organogel drug depot—to be both irrigation and migration resistant when subject to simulated in vivo conditions.

In a further experiment, pieces of the chicken thigh tissue that had been covered with the organogel drug depot (muscle fascia, see FIG. 10D top left, hypodermis, see FIG. 10D top right) were examined for release of the toluidine blue O dye (representing hydrophilic active agent particles) from the chicken thigh tissue. Two coated pieces of chicken thigh tissue were submerged in containers holding phosphate buffered saline (PBS). The PBS was exchanged hourly for four hours. The experiment showed that the organogel matrix continued to adhere to the chicken thigh tissue and did not penetrate into the muscle tissue or fascia, further supporting the migration resistant nature of the material. However, toluidine blue O dye was released into the buffer at each time point, and the released toluidine blue O dye penetrated both the muscle and skin tissue (see bottom FIG. 10D).

Melt Reconstitution

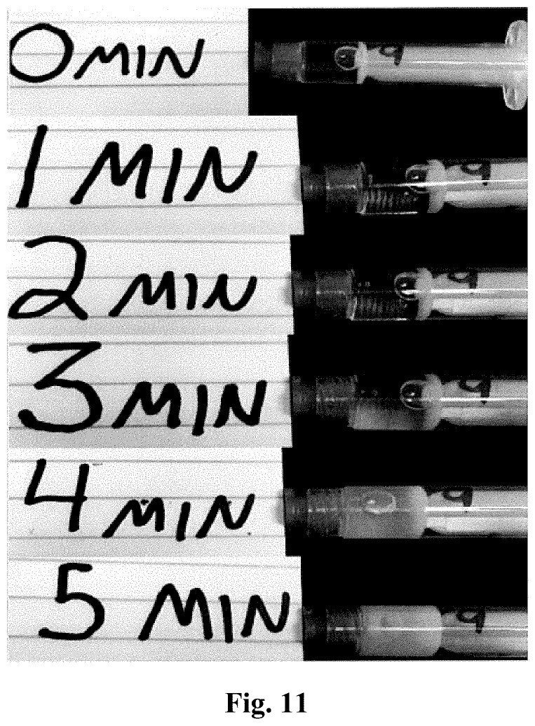

An organogel matrix formulation of 45:55 sorbitan monostearate:linoleic acid was prepared and heated to achieve a molten state. The molten organogel matrix was loaded into a syringe and allowed to cool to room temperature. Its appearance was observed at one minute intervals until the matrix was visually observed to reform into a solid/semisolid state. As shown in FIG. 11, the organogel matrix returned to a solid/semisolid state within approximately 5 minutes.

Heat Energy Analysis

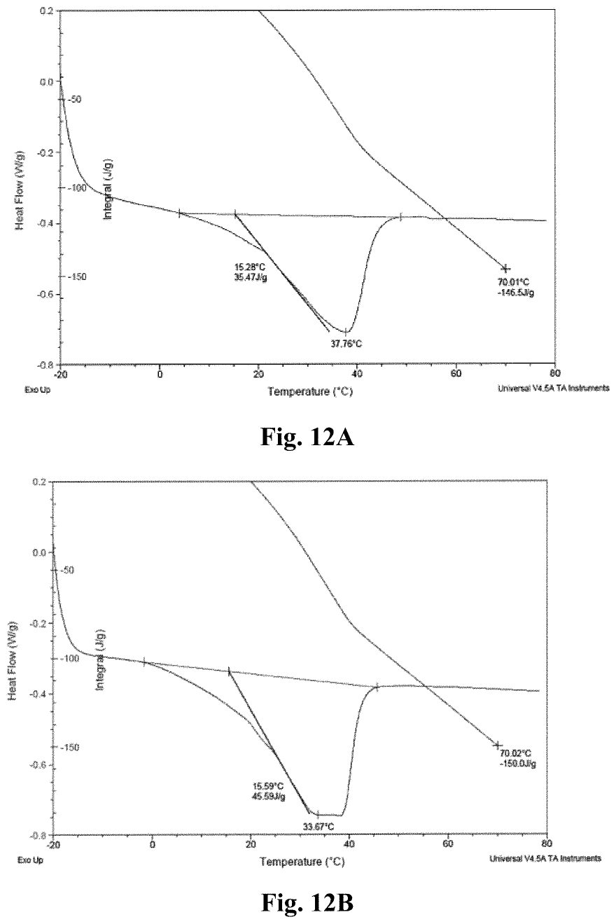

In order to determine the total amount of heat required to transform the organogel matrix into a molten state, two organogel matrix formulations were prepared; one, a base formulation of 45:55 sorbitan monostearate:linoleic acid, and a second including the base formulation with the addition of excipients, 5% PEG_{10 k} 0.5% Pluronic F127. Each sample was measured in a differential scanning calorimeter (DSC) from −20° C. to 80° C. The resultant graphs of the scans are shown in FIGS. 12A-B, respectively. The results indicate that from room temperature (approx. 20° C.) to above melting temperature (approx. 70° C.) requires about 150 J/g. This value is well within the limits produced by commercially available battery powered heaters, and which can be utilized, for example, in the heating devices as shown and described herein.

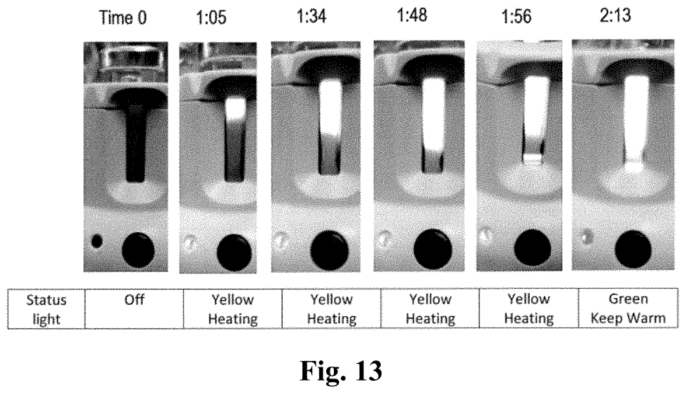

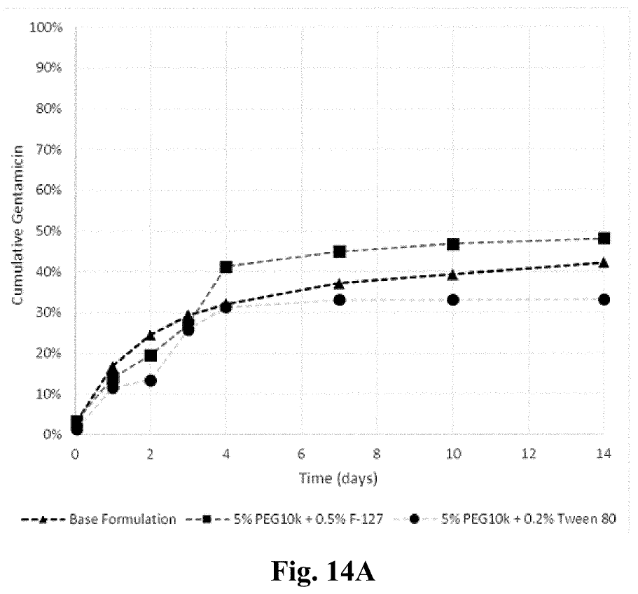

As an example, a battery-powered microprocessor-controlled device according to the embodiment shown in FIG. 7 was utilized to melt 6 grams of 45:55 sorbitan monostearate:linoleic acid with 5% PEG_{10 k} and 0.5% Pluronic F127. As can be seen in FIG. 13., the melting chamber was backlit, permitting visual observation through container 35 of melting as indicated by light passing through the container 35 holding the molten organogel matrix. Full melt was achieved in approximately 2 minutes. Thermal control is not limited to microprocessor control, and could be achieved through a variety of means, including, but not limited to, electromechanical thermostats, electronic thermostats or the use of positive temperature coefficient heating elements. Alternatively, the heating could be achieved through exothermic chemical reaction including, but not limited to, the oxidation of pure iron to iron oxide.

Method for in vitro elution from organogel gentamicin formulations

To evaluate the in vitro release of gentamicin sulfate from organogel formulations, approximately 193 mg of organogel-gentamicin sulfate formulation was loaded into a 13 mm diameter depression in a stainless steel disc and placed in a jar with 60 mL of phosphate buffered saline at 37° C. The buffer was sampled at 1 hour, and 1, 2, 3, 4, 7, 10 and 14 days. Complete buffer exchange was performed at all timepoints except 1 hour. Each eluent sample was briefly vortexted to ensure the sample was homogenous. Then, 1 mL of each eluent sample and corresponding blank was transferred to a separate 15 mL sterile tube. An equal volume of ethyl acetate was then added to each tube and then the tubes were either vortexed or manually shaken for about 10 seconds. The tubes were then placed on a test-tube rack and the layers were allowed to separate undisturbed for 10-15 minutes. The top layer containing any organogel components dissolved in the ethyl acetate layer was then carefully removed with a micropipette tip. An additional volume of ethyl acetate was then added to the tube and the extraction was repeated again to remove any additional organogel or excipients from the aqueous layer. The extracted aqueous bottom layer that contained gentamicin sulfate was then derivatized for quantification by UV absorbance. The derivatization reaction involved the reaction of the three primary amine groups on gentamicin with o-phthaladehyde (OPA) under basic conditions to form UV-absorbing fluorophores. Briefly, 1 mL of either the blank (usually phosphate buffered saline (PBS)) or extracted sample was added to a 15 mL sterile tube. To this, 500 μL isopropyl alcohol (IPA) and 150 μL of basic OPA was added to each tube that was then vortexed to mix. The tubes were then covered with foil for 15 minutes to allow the derivatization reaction to proceed at room temperature. Each sample was then transferred to a disposable plastic cuvette and the absorbance of the sample and blank was measured on a spectrophotometer at 332 nm. Quantification of gentamicin sulfate was then determined by interpolation from a standard curve constructed with gentamicin standards using Beer's law.

In vitro elution from syringe-to-syringe mixed organogel formulations

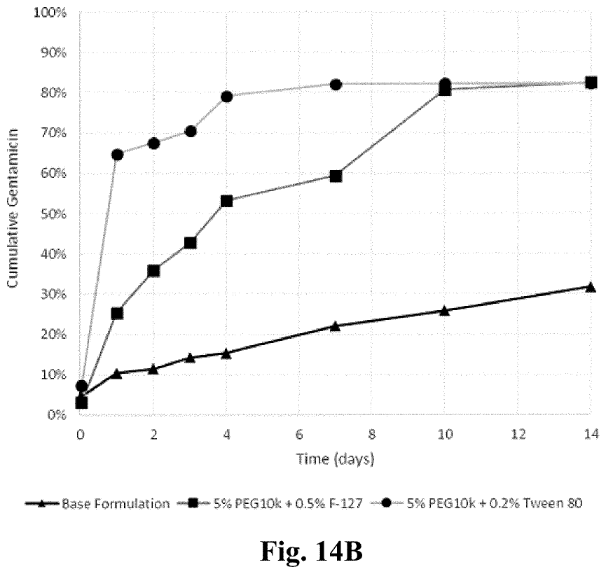

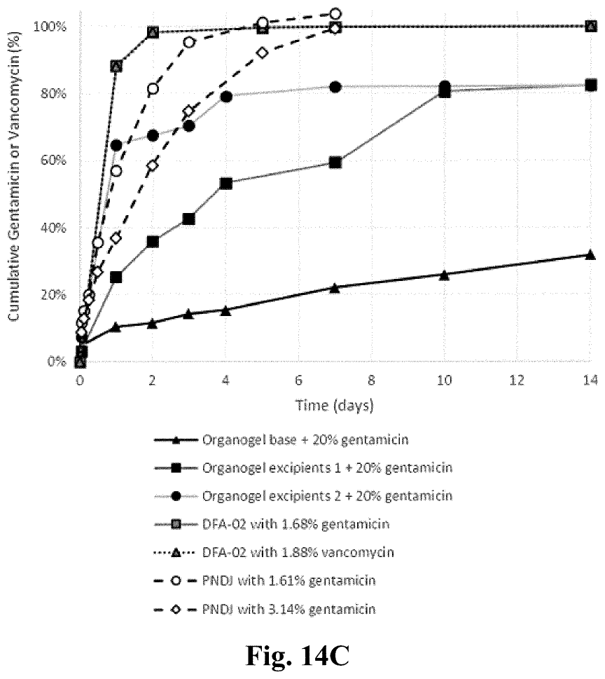

A 3 mL syringe of organogel formulation was loaded with approximately 930 mg of organogel formulation and a second syringe was loaded with micronized gentamicin sulfate equaling 20% of the organogel mass, approximately 187 mg. The micronized gentamicin sulfate was blended into the organogel by syringe-to-syringe mixing at room temperature. The organogel formulations consisted of a 45:55 sorbitan monostearate:linoleic acid base formulation and two additional formulations that included the base formulation plus excipients. One excipient formulation included a 5% PEG_{10 k} and 0.5% Pluronic F-127 excipient addition, and a second excipient formulation included 5% PEG_{10 k} and 0.2% Tween 80 excipient addition. The mixed formulations contained 16.7% gentamicin sulfate by mass. FIG. 14A illustrates the in vitro release of gentamicin sulfate from the organogel formulations with syringe-to-syringe mixing at room temperature. In the first day, 4-5 mg of gentamicin sulfate (12-17%) was released from the organogel-gentamicin sulfate formulations with 8-9 mg (26-29%) released through day 3. A lower rate of release was observed from day 4 through day 14, reaching a total percent observed in the buffer of approximately 41% cumulative gentamicin sulfate. Of note, the release of hydrophilic gentamicin sulfate from the organogel formulations was controlled without noted burst release; gentamicin sulfate release at 1 hour was between 0.4 and 1.1 mg (1-3%).

In vitro elution from melt-mixed organogel formulations

A 3 mL syringe of organogel formulation was loaded with approximately 947 mg of grease formulation and a glass vial was loaded with micronized gentamicin sulfate equaling 20% of the organogel mass, approximately 189 mg. The organogel formulation was injected into the glass vial using a vial adapter. The vial was placed into a water bath to melt the organogel. The vial was then shaken to suspend the gentamicin sulfate particles in the molten organogel, and the organogel plus gentamicin sulfate was drawn back into the syringe to cool and form into semisolid formulations of organogel plus gentamicin sulfate. The melt-mixed formulations contained 16.7% gentamicin sulfate by mass. As above, the organogel formulations consisted of a 45:55 sorbitan monostearate:linoleic acid base formulation and same two excipient formulations, base formulation plus 5% PEG_{10 k} and 0.5% Pluronic F-127 and base formulation plus 5% PEG_{10 k} and 0.2% Tween 80.

FIG. 14B illustrates in vitro release of gentamicin sulfate from melt-mixed organogel formulations. The use of melt-mixing enabled a range of gentamicin sulfate release rates from organogel formulations. In the first day, the base formulation released 3.3 mg (10%) of its gentamicin sulfate, while the excipient formulations released 8.2 mg (25%) and 20.8 mg (65%) gentamicin sulfate in one day. As above, no notable burst release was observed with 3-7% gentamicin sulfate release in one hour. The base formulation released 32% of its gentamicin sulfate load in a linear fashion over 2 weeks. The 5% PEG+0.5% F-127 formulation released 53% of its gentamicin in 4 days, and 81% within 10 days. The 5% PEG+0.2% Tween 80 formulation released 65% of its gentamicin sulfate in the first day and 79% by 4 days. The release curves of FIG. 14B demonstrate the ability to “tune” the organogel matrix by blending with excipients that increase water penetration into the matrix and dissolution of the therapeutic molecule and matrix. The melt-mixed formulations provided a greater range of release rates, with lower cumulative release of gentamicin sulfate from the base formulation in the melt-mixed form versus the room temperature mixed example, while simultaneously demonstrating faster release of the gentamicin sulfate from the excipient formulations in the melt-mixed examples versus the room temperature mixed examples.

Organogel v. Hydrogel in vitro antibiotic elution

Gentamicin sulfate release from the three melt-mixed organogel formulations described above and shown in FIG. 14B were compared against published literature values for several hydrogel drug depots. Release data was available for the following hydrogel drug depots: Dr. Reddy's DFA-02 formulated with 1.68% gentamicin plus 1.88% vancomycin (Penn-Barwell J G, Murray C K, and Wenke J C, J Orthop Trauma 2014; 28:370-375) and Sonoran Biosciences PNDJ formulated with either 1.61% gentamicin or 3.14% gentamicin (Overstreet D, McLaren A, Calara F, Vernon B, and McLemore R, Clin Orthop Relat Res 2015; 473:337-347). As shown in the graph in FIG. 14C, the release of gentamicin and vancomycin from Dr. Reddy's DFA-02 was 88% complete in the first day and 98% complete by day 2. Sonoran's PNDJ formulations took 5-7 days to reach approximately 100% release, with 59% or 81% released by day 2. In contrast, the base organogel formulation released only 11% of its gentamicin by day 2 and 22% in the first week. The addition of excipients was able to bring the two-day release to either 36% or 68%. This comparison demonstrates that organogels may achieve greater duration of drug release than achieved with hydrogels, and release rates are tunable by the selection of appropriate excipients.

Hydrophobic v hydrophilic in vitro elution profiles

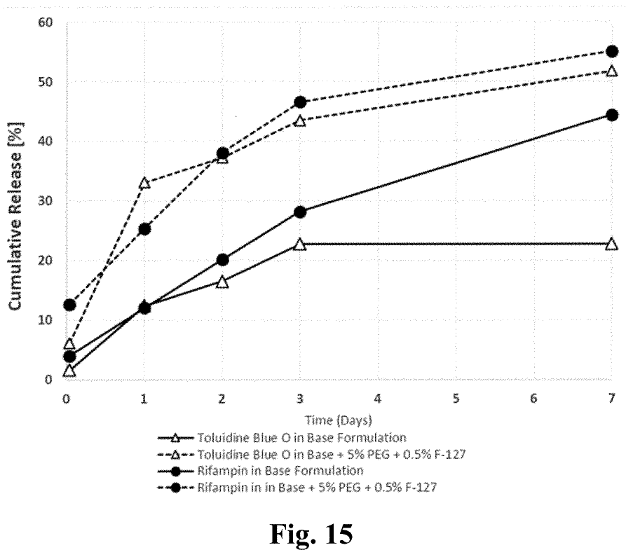

Two organogel matrix base formulations having 45:55 sorbitan monostearate:linoleic acid compositions were prepared by physical syringe-to-syringe mixing at room temperature in the semisolid state. One organogel matrix formulation included a 10% by weight addition of toluidine blue O dye to simulate a hydrophilic active agent. The other organogel matrix formulation included 10% by weight of rifampin, a relatively more hydrophobic active agent. Two additional organogel matrix excipient formulations were prepared with the base formulations previously described and including the addition of 5% PEG_{10 k} and 0.5% Pluronic F-127. The formulations were then placed into a 13 mm diameter depression in a stainless steel disc and placed in a jar with 60 mL of PBS plus 20% fetal bovine serum at 37° C., and their respective active agent elution profiles were measured. At each time point, the color of eluent was compared to visual standards prepared of 0, 1, 2.5, 3.75, 5, 7.5, 10, 15, 20, 30, 40, and 50 ppm of rifampin or toluidine blue O dye in PBS plus 20% fetal bovine serum. As shown in the graph at FIG. 15, each pair (i.e., base and excipient formulations) of organogel drug depots released their active agents at similar rates. In the first 3 days, the excipient-containing formulations eluted approximately 45% of their active agents, while the base formulations eluted approximately 25% of their active agents. At 7 days, both excipient formulations eluted approximately 53% of their active agents, while there was a deviation between the release of rifampin and Toluidine Blue O between days 3 and ∂in the base formulation. The rifampin sample reached 44%, while the toluidine blue O sample remained at 23%. Thus it can be seen the organogel matrix formulations can elute two dissimilar active agents at similar rates over a one week period into serum-containing buffer.

Furthermore, because the organogel matrix of the present disclosure has sparing water solubility due to the hydrophobic nature of its composition, the active agent particles' elution is limited by water availability for dissolution (irrespective of either a hydrophilic or hydrophobic active agent), followed by diffusion through the hydrophobic matrix. As previously discussed above, significant disadvantages are associated with hydrogel drug depots such as DAC-Gel, Dr. Reddy's DFA-02, Sonoran PNDJ, and Poloxamer 407 thermoreversible hydrogels. These exemplary hydrophilic drug depots are water-rich environments where the drug is in its soluble form, and release is only limited by diffusion through the water-rich network. As a result, hydrogel matrices are unable to achieve the long release durations and high drug loading ratios of the organogel matrices described herein. An additional benefit of the limited water availability within the organogel matrix is the relative stability of the active agent within the depot. Where the active agent is in particulate form, it has limited susceptibility to chemical reactions associated with degradation. Furthermore, the dissolution-limited approach enables both hydrophobic and hydrophilic molecules to be released at similar rates.

Antibacterial efficacy versus Staphylococcus aureus biofilm

Four sets of standard stainless steel trauma plates were colonized with Staphylococcus aureus while rolling in an inoculum of 10⁵ CFU/mL in 0.3% tryptic soy borth (TSB) in 15 mL tubes over 4 hours. The inoculated plates were placed into a lateral flow cell with intermittent 0.3% TSB medium replenishment every 4 hours with no flow between feedings.

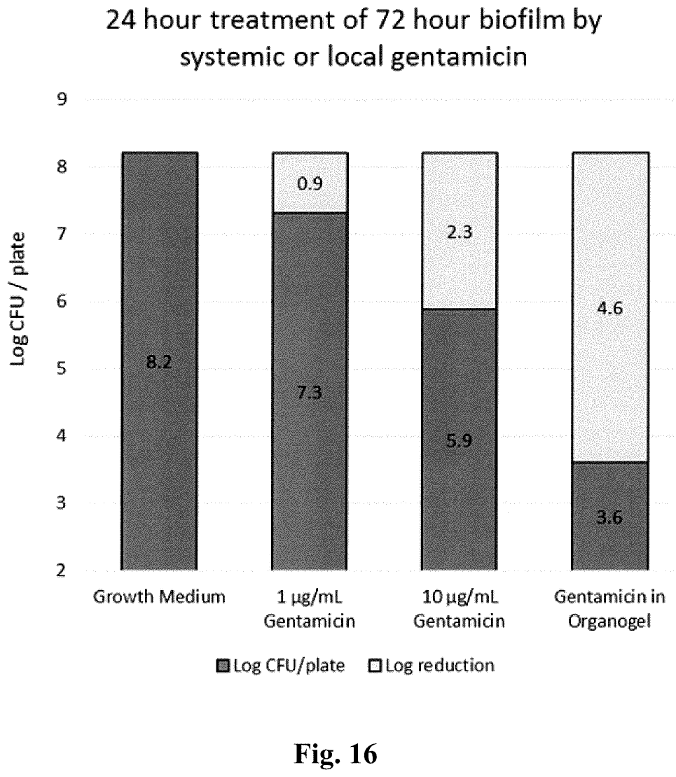

Biofilm growth proceeded in 0.3% TSB medium at 37° C. for 3 days to produce a mature biofilm. Each plate was rinsed twice in PBS, then returned to a sterile lateral flow cell for 1 day of treatment. One set of plates served as a control group, fed with 0.3% TSB growth medium. The second set was treated with 0.3% TSB plus 1 μg/ml gentamicin sulfate. The third set was treated with 0.3% TSB plus 10 μg/m1 gentamicin sulfate. These concentrations represent a range of clinically-relevant blood levels for systemic administration of gentamicin sulfate, here provided as a supplement to the 0.3% TSB medium. The fourth group consisted of a 590 mg organogel drug depot placed into the growth chamber without contacting the trauma plate with adhered bacterial biofilm. The organogel drug depot included 16.7% by weight of gentamicin sulfate melt-mixed with 45:55 sorbitan monostearate:linoleic acid (corresponding to a 1:5 weight ratio of drug:organogel matrix) with the addition of 5% PEG_{10 k} and 0.5% Pluronic F-127 as excipients. This group was fed with 0.3% TSB growth medium without any antibiotics. In all four sets, the culture medium was exchanged once every four hours by lateral flow for four minutes. Note that the gentamicin sulfate released from the organogel formulation inside the growth chamber was rinsed away every four hours, requiring additional gentamicin sulfate to elute from the formulation to continue antibacterial activity. As shown in FIG. 16, gentamicin sulfate released from the organogel drug depot was more effective against a 3-day S. aureus biofilm grown on a trauma plate than systemic delivery of gentamicin sulfate. Importantly, even though the second and third sets of implants were continuously exposed to clinically-relevant concentrations of gentamicin sulfate over 24 hours, the organogel drug depot showed higher effectiveness in killing bacteria in the biofilm despite the gentamicin sulfate being rinsed away every four hours.

What is claimed:

1. A method of delivering an active agent to a non-sterile open wound site comprising:
compounding solid particles of an active agent within a biocompatible organogel matrix comprising an organogelator and a biocompatible organic solvent to form an organogel drug depot; and,
delivering the organogel drug depot to a non-sterile open wound site, wherein at the time of delivery the open wound site includes soft tissue, hard tissue, or both, that are exposed to a non-sterile environment;
wherein the step of compounding and the step of delivering are performed contemporaneously; and,
wherein the organogel is in a solid or semisolid state during the step of delivering.

2. The method of claim 1, wherein the contemporaneous compounding and delivering are within 1.5 hours or less of each other.

3. The method of claim 2, wherein the contemporaneous compounding and delivering are within 1.0 hours or less.

4. The method of claim 2, wherein the contemporaneous compounding and delivering are within 0.5 hours or less.

5. The method of claim 1, wherein the organogel matrix is configured to adhere to the soft tissue, hard tissue, or both, in a substantially aqueous environment

6. The method of claim 1, wherein compounding comprises heating the organogel matrix to melt the matrix and incorporating the solid particles into the melted matrix.

7. The method of claim 6, wherein the method further comprises, after incorporating the solid particles, cooling the melted matrix to form the organogel drug depot.

8. The method of claim 7, wherein cooling the melted matrix is within about 10 minutes or less.

9. The method of claim 1, wherein compounding comprises a physical mixing between the organogel matrix in solid or semisolid state and the solid particles.

10. The method of claim 1, wherein the organogel matrix has a melting point above 37° C.

11. The method of claim 1, wherein the biocompatible organic solvent has a melting point below 20° C.

12. The method of claim 1, wherein the solid particles are disposed within the biocompatible organic solvent.

13. The method of claim 1, wherein the organogel matrix has a solubility in water of less than 1 g/L.

14. The method of claim 1, wherein the organogelator comprises one or more fatty acids, or salts or esters of fatty acids, and mixtures thereof.

15. The method of claim 14, wherein the fatty acid ester is sorbitan monostearate.

16. The method of claim 1, wherein the biocompatible organic solvent is a plant or animal derived oil, or a synthetic derivative thereof.

17. The method of claim 16, wherein the oil comprises one or more fatty acids.

18. The method of claim 17, wherein the one or more fatty acids comprises triglycerides.

19. The method of claim 17, wherein the one or more fatty acids comprises linoleic acid.

20. The method of claim 1, wherein the active agent is an antimicrobial agent, an antibiotic agent, or a local anesthetic agent, or a combination thereof.

21. The method of claim 20, wherein the active agent is an antimicrobial agent.

22. The method of claim 20, wherein the active agent is gentamicin, vancomycin, ertapenem, or tobramycin.

23. The method of claim 20, wherein the active agent is a local anesthetic agent.

24. The method of claim 1, wherein the active agent is soluble, freely soluble, or very soluble in water.

25. The method of claim 1, wherein the active agent is sparingly soluble, slightly soluble, very slightly soluble, or insoluble in water.

26. The method of claim 1, wherein the solid particles have a D(50) median particle size in the range of 1 μm to about 1 mm.

27. The method of claim 1, wherein the weight ratio of organogelator to biocompatible organic solvent in the organogel matrix is in the range of about 5:95 to about 70:30.

28. The method of claim 1, wherein the organogel matrix further comprises one or more excipients.

29. The method of claim 28, wherein the one or more excipients includes biocompatible surfactants or biocompatible hydrophilic small molecules, or a combination thereof.

30. The method of claim 28, wherein the one or more excipients includes Poly(ethylene glycol) (PEG), Pluronic F127, Tween 80, or a mixture of any combination thereof.

31. The method of claim 1, wherein the organogel drug depot is delivered to the open wound site by injection from a syringe through a percutaneous needle or cannula.

32. A method of delivering an active agent to a surgical site comprising:
perioperatively compounding solid particles of an active agent within a biocompatible organogel matrix to form an organogel drug depot configured for controlled release; and
intraoperatively delivering the organogel drug depot to the surgical site;
wherein the organogel matrix comprises an organogelator and a biocompatible organic solvent, and wherein the organogel drug depot is in a solid or semisolid state during the step of intraoperative delivery.

33. A system for preparing an organogel drug depot for local delivery to a surgical site comprising:
an organogel matrix comprising an organogelator and a biocompatible organic solvent;
an active agent comprising solid particles;
a container including at least one wall having an outer surface, the container defining a volume capable of containing the organogel matrix and active agent solid particles; and
a heating component configured to contact the outer surface and supply an amount of heat to the container.
